(19) United States
(12) Patent Application Publication
(10) Pub. No.: US 2025/0195912 A1
(43) Pub. Date: Jun. 19, 2025

(54) LIGHT THERAPY DEVICE STAND

(71) Applicant: Mark Sawyer, Lake Mary, FL (US)

(72) Inventor: Mark Sawyer, Lake Mary, FL (US)

(21) Appl. No.: 18/945,285

(22) Filed: Nov. 12, 2024

Related U.S. Application Data
(63) Continuation of application No. 18/643,452, filed on Apr. 23, 2024.
(60) Provisional application No. 63/612,237, filed on Dec. 19, 2023.
(60) Provisional application No. 63/612,108, filed on Dec. 19, 2023.
(60) Provisional application No. 63/612,118, filed on Dec. 19, 2023.

(51) Int. Cl. A61N 5/06

(52) U.S. Cl. CPC A61N 5/0613; A61N 5/0616; A61N 5/0617; A61N 5/0618; A61N 2005/0626; A61N 2005/0652; A61N 2005/0659; A61N 2005/0663

(57) ABSTRACT

A light therapy device stand includes a base, a telescoping extension arm, a receptacle connected to the extension arm, an elongated support bar, a plurality of sliding connectors, a plurality of connection rails, and a plurality of extension rails. Individual light therapy devices can be secured onto the support bar via the sliding connectors. Standard sized light therapy devices can be secured onto the support bar in a stacked configuration via the connection rails, and non-standard sized light therapy devices can be secured onto the support bar in a stacked configuration via the extension rails. The support bar rotates 90 degrees relative to the extension arm to transition each connected light therapy device between a vertical orientation and a horizontal orientation.

### CROSS-REFERENCE TO RELATED APPLICATIONS

This application claims the benefit of U.S. Application Ser. No. 63/612,108 filed on Dec. 19, 2023, U.S. Application Ser. No. 63/612,118 filed on Dec. 19, 2023, and U.S. patent application Ser. No. 18/643,452 filed on Apr. 23, 2024, the contents of each of which are incorporated herein by reference.

### TECHNICAL FIELD

The present invention relates generally to the field of light therapy devices, and more particularly to a stand for positioning one or more light therapy devices in a specified orientation.

### BACKGROUND

The statements in this section merely provide background information related to the present disclosure and may not constitute prior art.

Scientific research has shown that exposing human skin to certain types of light can have a meaningful effect on the person's overall health. Depending on the intensity and spectrum/wavelength of the light, these effects can include reducing stress, eliminating headaches, promoting hair growth, increasing collagen production, reducing the appearance of acne, and even fat reduction.

Many commercially available light therapy devices are manufactured to include an industry standard height/length of about 19 inches and a width of about 8-10 inches so as to be suitable for providing light therapy to a single area of a user's body such as the head, chest, or back, for example. In this regard, it is important that a user be able to accurately position the device to ensure the desired portion of their body receives the treatment.

Most commercially available light therapy devices are provided with a mounting kit having a pair of posts that are screwed into the top surface of the device which engage a pair of elongated cords and a pulley system to allow the device to be suspended from a door or wall via a hook. However, if/when users seek to link multiple devices together in a side-by-side configuration, they must use two separate mounting systems. When the user seeks to link multiple devices together vertically, they must connect the panels to each other using the above noted posts and keyhole openings in the bottom of each device.

The present invention, directed to a light therapy device stand differs from the conventional art in several aspects. The manner by which will become more apparent in the description which follows, particularly when read in conjunction with the accompanying drawings.

### SUMMARY OF THE INVENTION

The present invention is directed to a light therapy device stand. One embodiment of the present invention can include a wheeled base having a center platform and a pair of side rails extending outward therefrom. A riser section can extend upward from the center platform and can be connected to an attachment assembly for removably connecting up to four light therapy devices in a plurality of different configurations.

In one embodiment, the riser section can include a pair of fixed bar members that are connected to the center platform, and a pair of sliding bar members that selectively raise and lower the attachment assembly relative to the wheeled base. The attachment assembly can include a pair of connection rails that can engage each of a single light therapy device, a pair of light therapy devices in a 2-high stacked configuration, and four light therapy devices in a 2-high and a 2-wide stacked configuration. The attachment assembly can also include a plurality of horizontal mounting brackets for securing the light therapy devices together when in the 2-wide stacked configuration.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is an exploded parts view of a light therapy device stand that is useful for understanding the inventive concepts disclosed herein.

FIG. 2 is a perspective view of the base of the light therapy device stand, in accordance with one embodiment of the invention.

FIG. 3A is a front view of the base and riser section of the light therapy device stand in the retracted position, in accordance with one embodiment of the invention.

FIG. 3B is a back view of the base and riser section of the light therapy device stand in the extended position, in accordance with one embodiment of the invention.

FIG. 4 is a perspective view of the base, riser section and mounting plate of the light therapy device stand, in accordance with one embodiment of the invention.

FIG. 5 is a perspective view of the connection rails of the light therapy device stand, in accordance with one embodiment of the invention.

FIG. 6A is a back view of the light therapy device stand in operation with a pair of light therapy devices stacked in a 2-high configuration, in accordance with one embodiment of the invention.

FIG. 6B is a front view of the light therapy device stand in operation with a pair of light therapy devices stacked in a 2-high configuration, in accordance with one embodiment of the invention.

FIG. 7A is a perspective view of the connection rails and the horizontal mounting plates of the light therapy device stand, in accordance with one embodiment of the invention.

FIG. 7B is a back view of the light therapy device stand in operation with a pair of light therapy devices stacked in a 2-wide configuration, in accordance with one embodiment of the invention.

FIG. 7C is a front view of the light therapy device stand in operation with a pair of light therapy devices stacked in a 2-wide configuration, in accordance with one embodiment of the invention.

FIG. 8A is another perspective view of the connection rails and the horizontal mounting plates of the light therapy device stand, in accordance with one embodiment of the invention.

FIG. 8B is a back view of the light therapy device stand in operation with a pair of light therapy devices stacked in a 2-wide and a 2-high configuration, in accordance with one embodiment of the invention.

FIG. 8C is a front view of the light therapy device stand in operation with a pair of light therapy devices stacked in a 2-wide and a 2-high configuration, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

### Definitions

As described herein, a “unit,” “assembly” and “section” mean a series of identified physical components which are linked together and/or function together to perform a specified function.

As described throughout this document, the term “about” “approximately” “substantially” and “generally” shall be used interchangeably to describe a feature, shape, or measurement of a component within a tolerance such as, for example, manufacturing tolerances, measurement tolerances or the like.

As described herein, the term “removably secured,” and derivatives thereof shall be used to describe a situation wherein two or more objects are joined together in a non-permanent manner so as to allow the same objects to be repeatedly joined and separated.

As described throughout this document, the term “complementary shape,” and “complementary dimension,” shall be used to describe a shape and size of a component that is identical to, or substantially identical to the shape and size of another identified component within a tolerance such as, for example, manufacturing tolerances, measurement tolerances or the like.

As described herein, the term “slidingly engage”, “telescopically connected” and derivatives thereof shall be used interchangeably to describe a situation wherein two or more identified objects are connected linearly such that one identified part slides into and out from the other identified part so as to selectively lengthen or shorten the total linear length of the assembly.

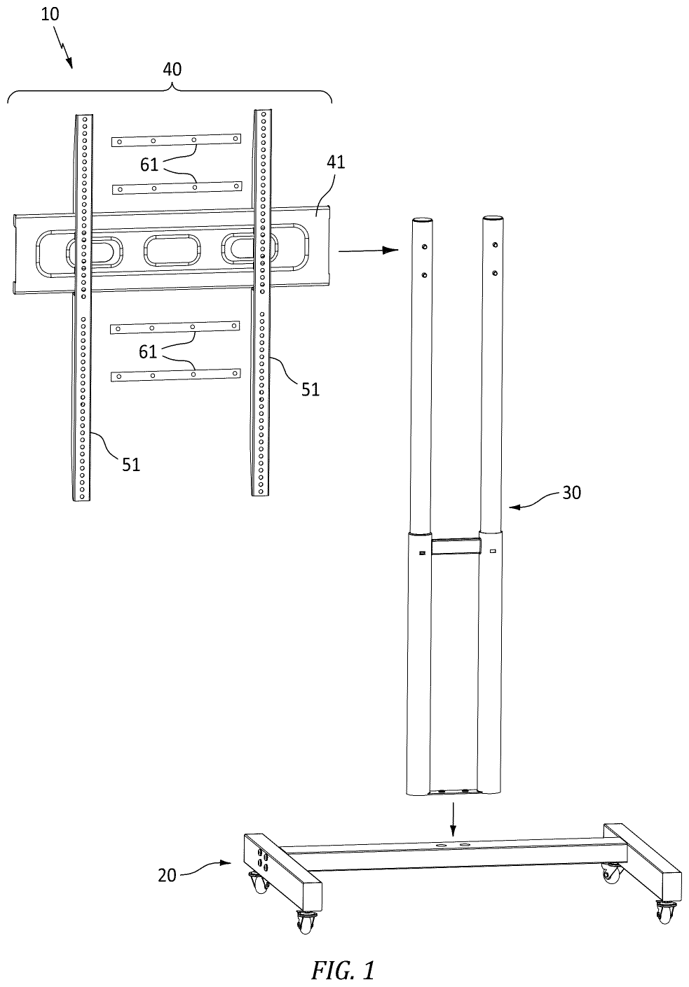

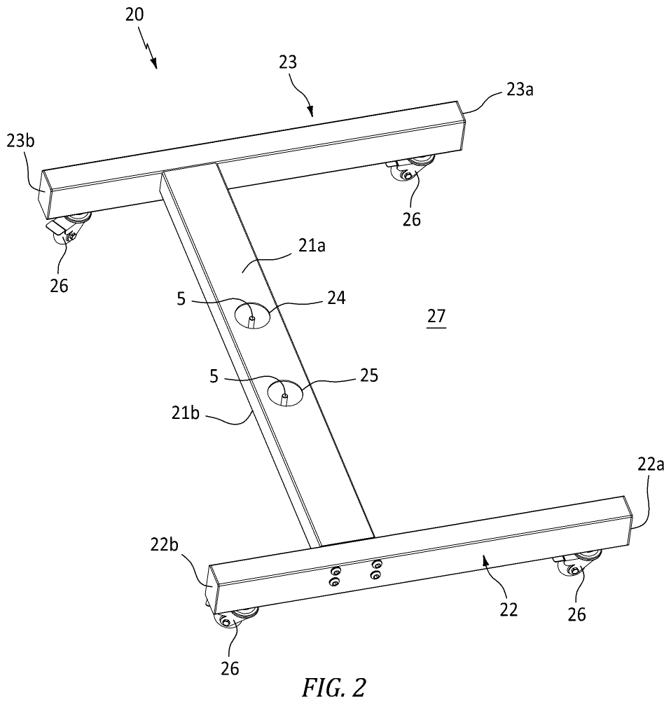

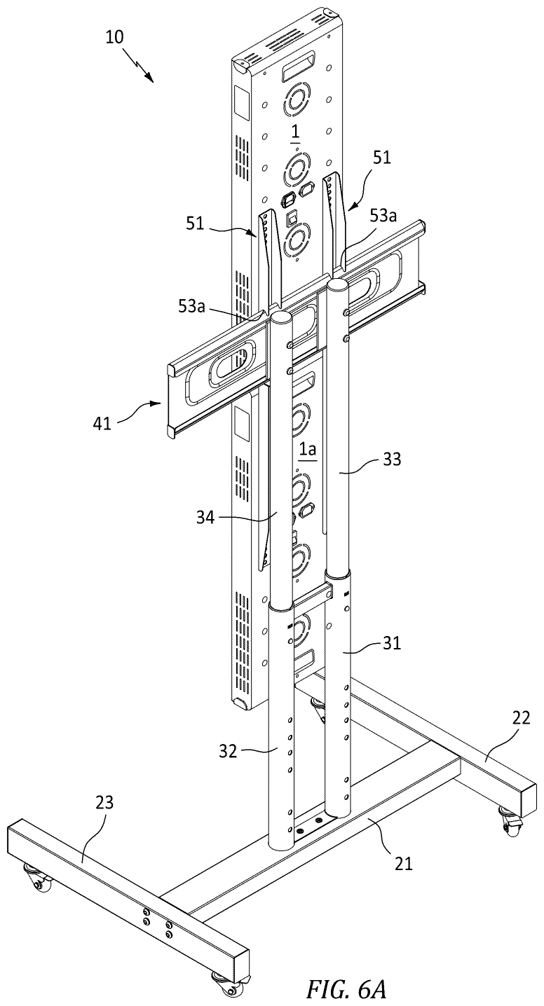

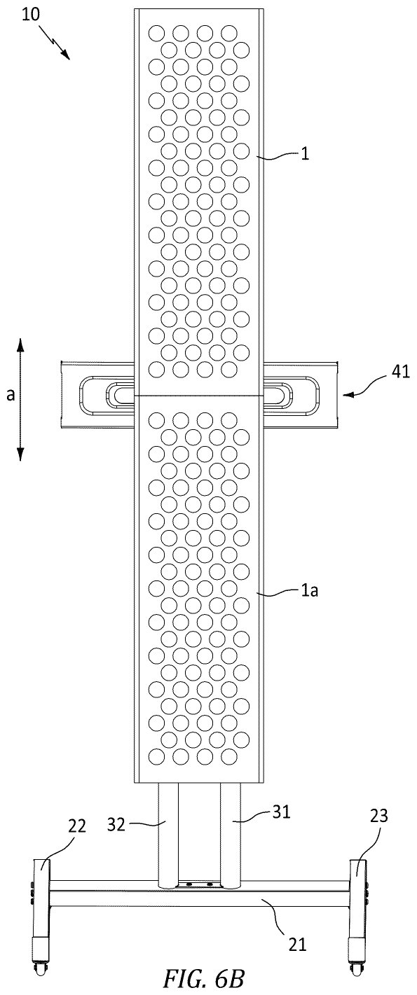

FIGS. 1-8C illustrate one embodiment of a light therapy device stand 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 6B.

As described herein, the inventive stand 10 can function to engage and position up to four light therapy devices such as those described in copending U.S. patent application Ser. No. 18/643,452 to Sawyer, the contents of which are incorporated herein by reference. However, other types of light therapy devices may also be used.

As shown in the drawings, one embodiment of the light therapy device stand 10 for engaging and positioning one or more light therapy devices 1 can include, essentially, a base 20, a riser section 30, and a panel attachment assembly 40.

The base 20 can form the bottom end of the stand 10 and can function to provide a stable platform from which the riser section and panel connection assembly can extend. In one embodiment, the base can include an elongated center rail 21 having a pair of perpendicularly oriented side rails 22 and 23 along each end.

In one embodiment, a first pair of openings 24 and 25 can be disposed along the top surface 21a of the center rail, and mounting hardware such as the illustrated bolts 5 or other such connectors can be positioned through the bottom surface 21b of the rail to engage the fixed bar members of the below described riser section. Likewise, a plurality casters 26 can be located along the bottom ends of both side rails 22 and 23.

As described herein, the base 20 may be formed from materials that are, for example, relatively strong and stiff for their weight. Several nonlimiting examples include but are not limited to various metals or metal alloys (e.g., aluminum, steel, titanium, or alloys thereof), plastic/polymers (e.g., high-density polyethylene (HDPE), rigid polyvinyl chloride (PVC), malleable polyethylene terephthalate (PET)), and/or various composite materials. Although illustrated with regard to a U-shaped base member shape, this is for illustrative purposes only, as any number of other configurations are also contemplated.

In one embodiment, the base member will be sized so as to permit users to mount up to four light therapy devices in a 2-high and 2-wide configuration (e.g., a 2×2 configuration). To this end, the center rail 21 will preferably include a length (e.g., distance between the side rails) of 28.5 inches so as to extend beyond the width of two light therapy devices positioned side by side. Additionally, each of the side rails 22 and 23 can include lengths (e.g., distance between front and back ends 22b-22c and 23b-23c) of about 46 inches with the center rail being located about 12 inches from the back ends 22b and 23b of the side rails. Such a feature provides ensures that up to four light therapy devices will be completely stable when secured onto the stand, and also creates an unobstructed treatment area 27 directly in front of the center rail and between the two side rails extending forward where a user can stand when operating the device.

The inclusion of the treatment area 27 is important, as users wear vision limiting eye protection when using light therapy devices and must position themselves inches from the devices when accessing the system controller(s) and/or receiving certain types of treatments. As such the treatment area provides a safe location where the user will not trip on the side rails which may be obscured when wearing the requisite eye protection.

Although described above with regard to a particular shape, size, or orientation, those of skill in the art will recognize that other shapes, sizes, and/or orientations of components are also contemplated.

The riser section 30 can allow a user to selectively raise and lower the height of panel assembly to a user-desired position. In one embodiment, the riser section can include a pair of fixed bar members 31 and 32 each having a bottom end 31a and 32a, elongated hollow middle portions 31b and 32b, and open top ends 31c and 32c.

In one embodiment, the bottom ends of the fixed bar members can have a cross-sectional shape and dimension that is complementary to the cross-sectional shape and dimensions of the first pair of openings 24 and 25 so as to be positioned therein. Moreover, threaded apertures 31a1 and 32a1 can be positioned along the bottom ends of the arm sections to engage and receive the above-described connectors 5 in order to secure the arm sections onto the base. Due to the weight of the panels to which the stand is designed to accommodate, one or more lateral braces 35 can be provided to secure the fixed bar members together, and to prevent the same from moving or swaying relative to the other.

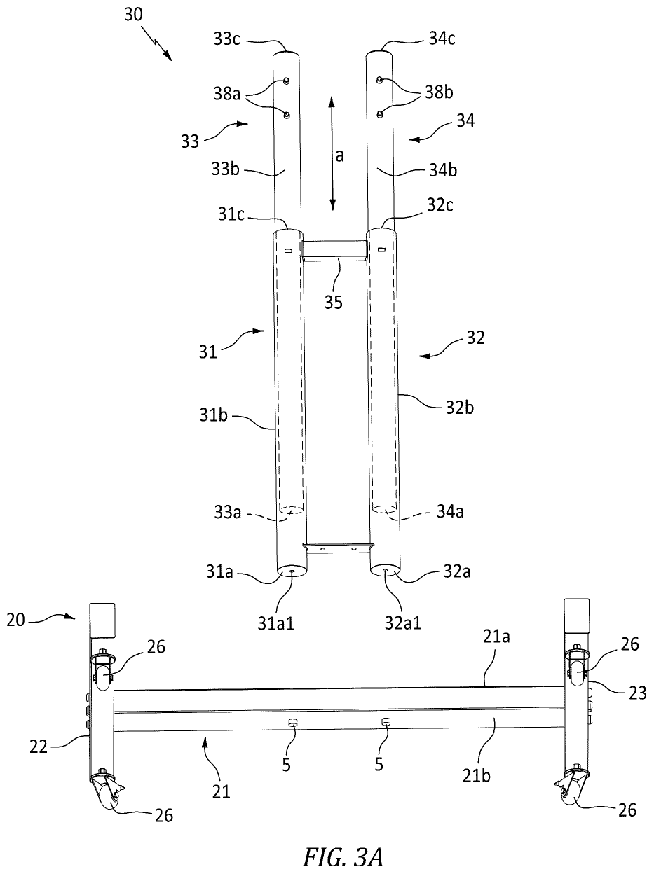

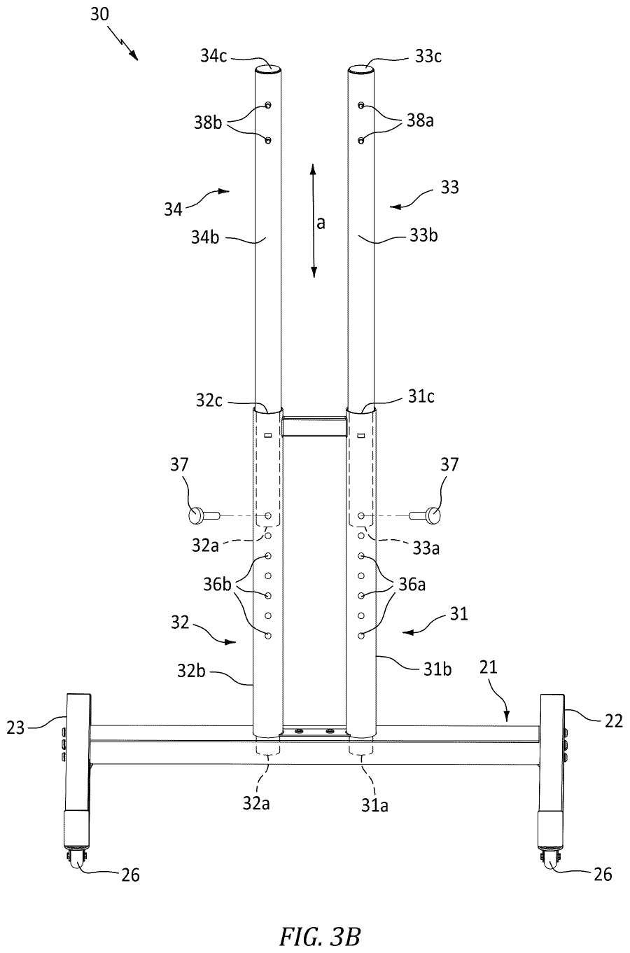

In one embodiment, the riser section 30 can also include a pair of sliding bar members 33 and 34. As shown by arrow a, the sliding bar members can each include a bottom end 33a and 34a, a middle portion 33b and 34b, and top ends 33c and 34c. In one embodiment, the bottom ends 33a and 34a are positioned through the open top ends 31c and 32c of the fixed bar members so as to telescopically extend from and retract into the hollow middle portions 31b and 32b, of the fixed bar members 31 and 32, respectively. In this regard, the sliding bar members can be positioned anywhere between the fully retracted position shown at FIG. 3A, and the fully extended position shown at FIG. 3B.

As shown best at FIG. 3B, each of the fixed bar members 31 and 32 can include a plurality of positioning openings 36a and 36b along the respective back surfaces. Each of the openings can be selectively aligned with a threaded aperture located along the lower portion of the sliding bar members 33 and 34, respectively, as the bars are moved up and down. When positioned at a desired height, a locking pin 37, bolt or other such connector can pass through the aligned openings to lock the riser section at the set height. Of course, any number of other mechanisms such as a tensioning ring, for example, can be provided to secure the sliding bar members at a user defined height.

In either instance, each of the fixed bar members and the sliding bar members may also be formed from materials that are, for example, relatively strong and stiff for their weight. Several nonlimiting examples include but are not limited to various metals or metal alloys, various plastics/polymers, and/or various composite materials.

As will be described and shown below, the attachment assembly 40 can include a mounting plate 41, a pair of connection rails 51, and a plurality of horizontal mounting brackets 61 which can function to engage and secure up to four individual light therapy devices onto the stand in a 2-high (e.g., vertically stacked) configuration, and/or a 2-wide (e.g., horizontally stacked) configuration.

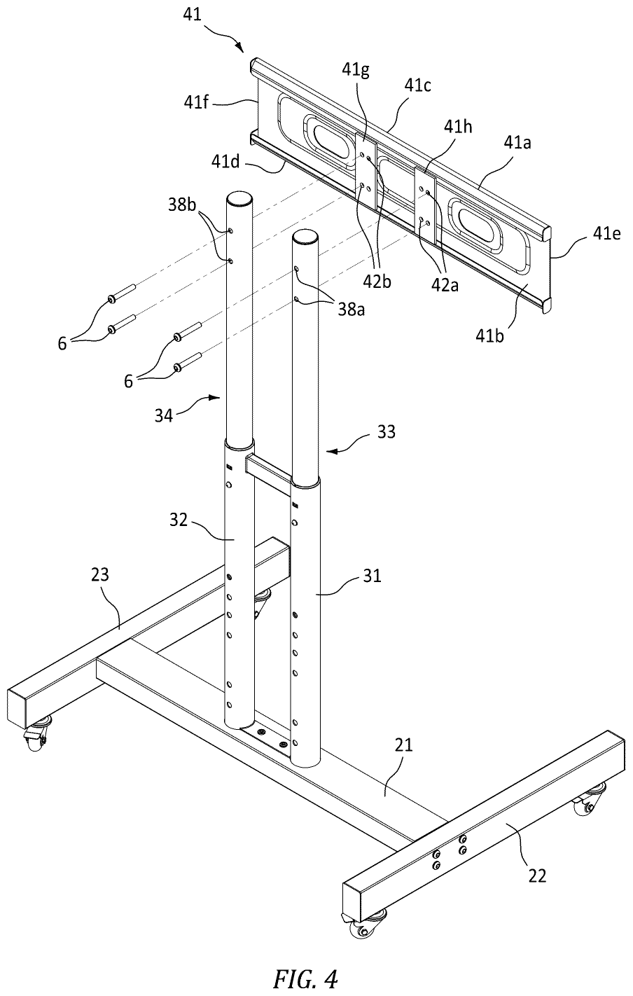

As shown at FIG. 4, the mounting plate 41 of the panel attachment assembly can be positioned onto the upper portions of the sliding bar members in order to engage and receive between 1 and 4 light therapy devices in a plurality of different configurations. In one embodiment, the mounting plate 41 can include an elongated, generally rectangular-shaped member having a front surface 41a, a back surface 41b, a top end 41c, a bottom end 41d and a pair of side surfaces 41e and 41f.

A pair of mounting surfaces 41g and 41h having a pair of threaded apertures 42a and 42b, respectively, can be positioned along the back surface 41b and can function to receive bolts 6 or other such mounting hardware that extends through mounting apertures 38a and 38b on the top ends of the sliding bar members 33 and 34, respectively.

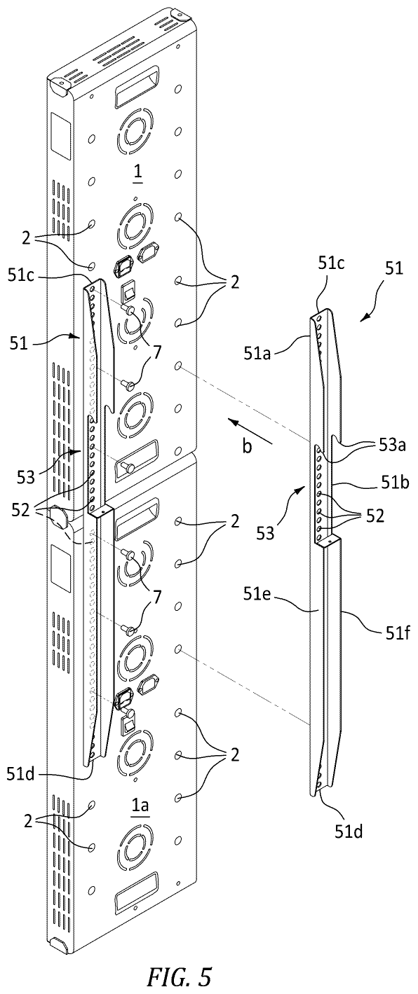

As shown at FIG. 5, each connection rail 51 can include an elongated member having a flat front surface 51a, a back surface 51b, a top end 51c, a bottom end 51d and a pair of side edges 51e and 51f that extend outward from the back surface. A plurality of openings 52 can be disposed along the length of the rails at locations complementary to the locations of the mounting apertures 2 located along the back surface of a single light therapy device 1, or two light therapy devices 1 and la that are stacked vertically in a 2-high configuration.

As shown by arrow b, each of the rails can be positioned onto the back surfaces of the light therapy device(s) and upon aligning the openings 52 with mounting apertures 2, bolts 7 or other such connectors can be passed therethrough to secure the rails onto the devices.

In one embodiment, each of the rails 51 can also include indentations 53 along each of the side surfaces. Additionally, each of the side rails can include protruding lips 53a extending into the indentations for engaging the top surface 41c of the mounting plate 41 to hang the vertically stacked panels 1 and 1a on the stand as shown at FIGS. 6A and 6B. Such a feature also allows the rails and devices to move laterally (e.g., toward, and away from the sides of the panel 41).

In the preferred embodiment, the connection rails will be provided in a length (e.g., distance between top end 51c and bottom end 51d) of about 17 inches and about 34 inches. Such dimensions are important to allow the rails to be connected onto small light therapy devices such as the BioMax 300 lights commercially available by Platinum LED therapy lights®, having individual heights of about 18 inches each, and large light therapy devices such as the BioMax 900 lights commercially available by Platinum LED therapy lights®, having individual heights of about 36 inches each without causing the rails to extend above or below the devices.

Such a feature is important to prevent consumers from becoming injured by the exposed rails during operation of the stacked devices, and to prevent the devices from moving or shifting relative to each other which could result in uneven alignment with a patient's body. Of course, the rails can be manufactured in other dimensions so as to engage two stacked light therapy devices having different dimensions.

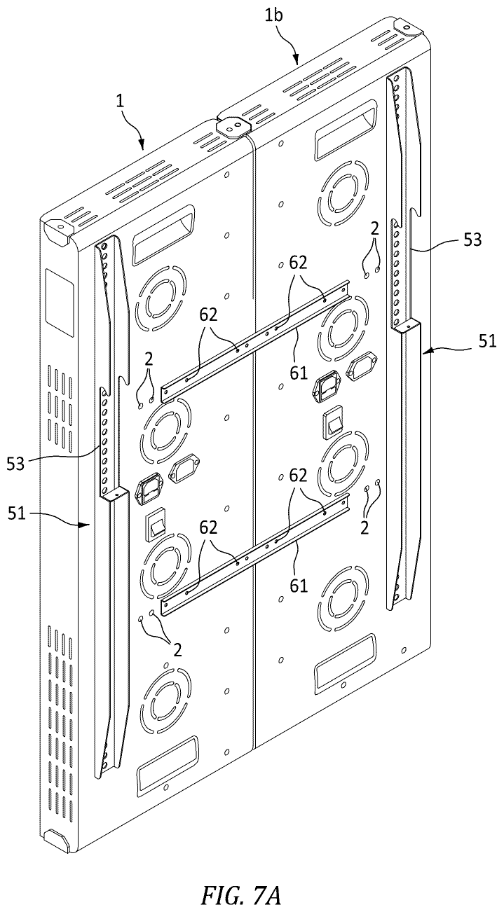

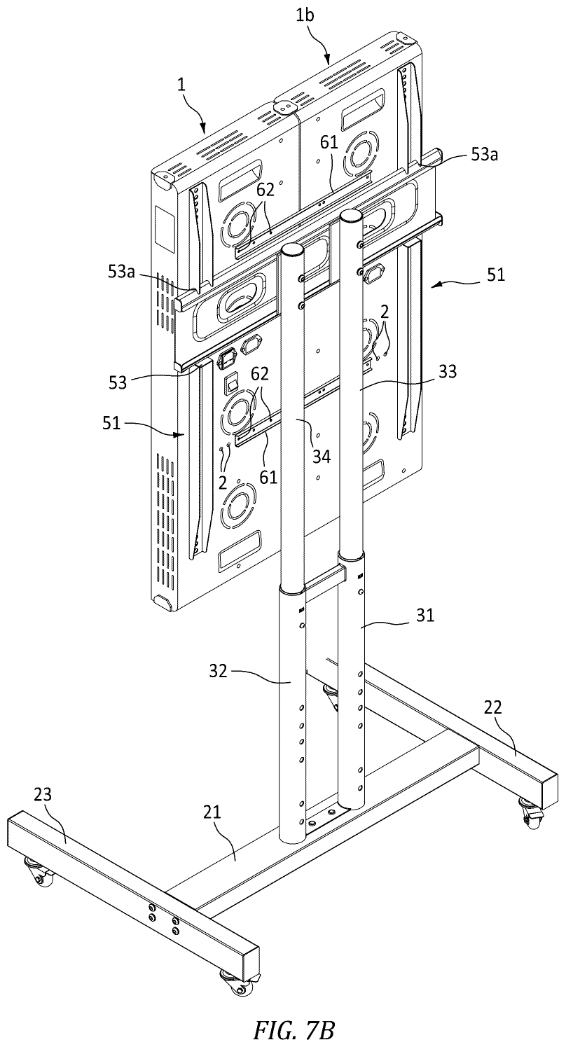

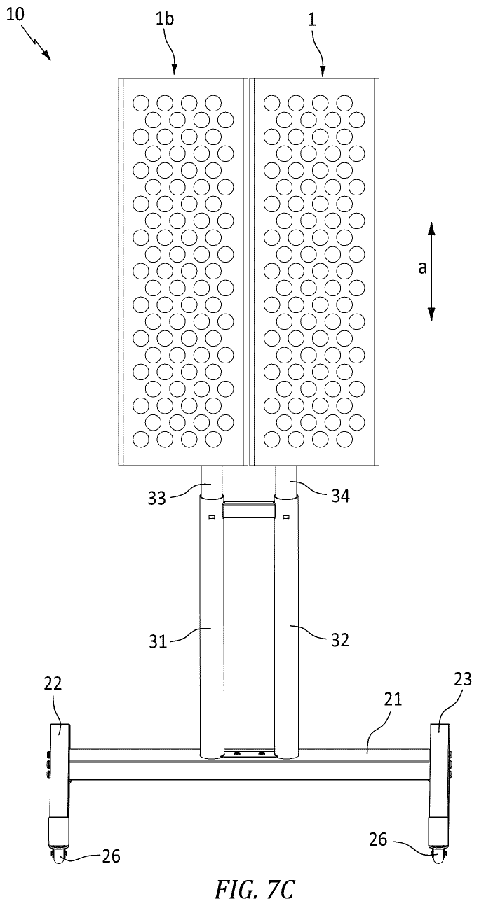

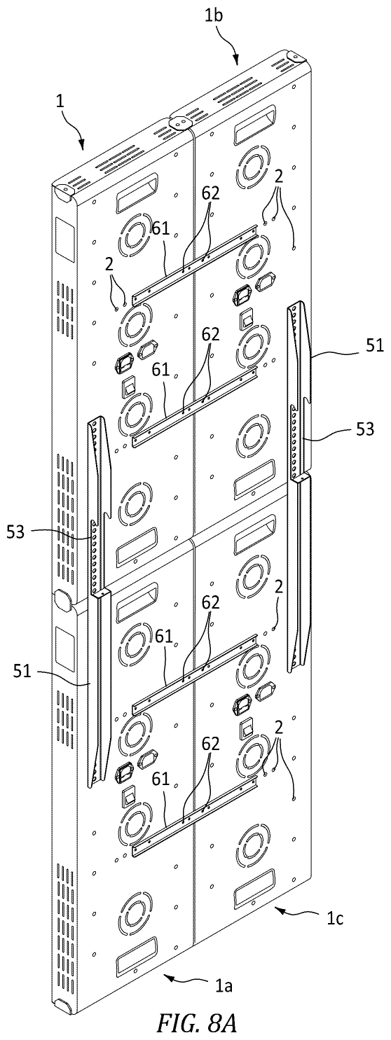

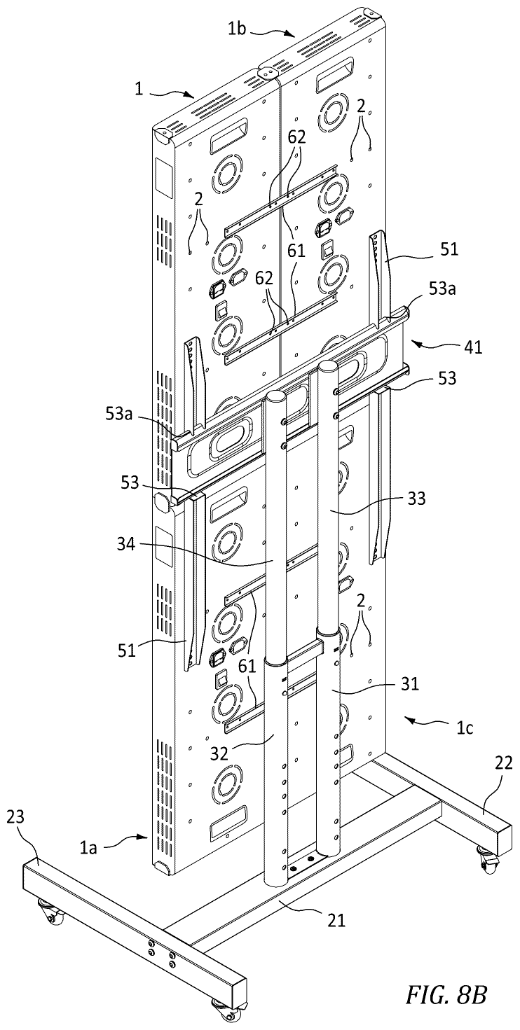

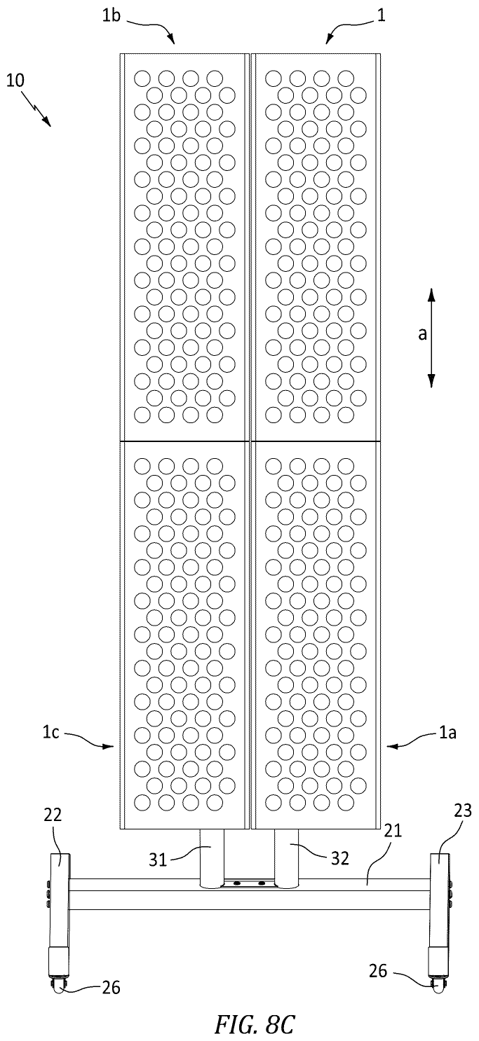

As shown at FIGS. 7A and 8A, the horizontal mounting brackets 61 can function to engage and secure two light therapy devices together horizontally in a 2-wide configuration. As such, each bracket can include an elongated generally planar member having a plurality of openings 62 disposed along the length thereof. Each of the openings 62 can be positioned at locations complementary to the locations of the mounting apertures 2 located along the back surface of two light therapy devices positioned side by side. In this regard, the two devices can be secured together by aligning the openings 62 of the brackets 61 with the mounting apertures 2 on each device and passing bolts or other such connectors therethrough.

As noted above, the stand is designed to accommodate up to four light therapy devices utilizing the horizontal mounting brackets 61 to connect the devices together horizontally in a 2-wide configuration, and the connection rails 51 to connect the devices together vertically in a 2-high configuration.

As such, the connection rails 51 can be connected to the back surfaces of two light therapy devices 1 and 1b as shown at FIG. 7A to secure the two light therapy devices onto the stand in a 2-wide configuration as shown at FIGS. 7B and 7C. Additionally, the connection rails 51 can be connected to the back surfaces of four light therapy devices 1, 1a, 1b and 1c, as shown at FIG. 8A to secure the four light therapy devices onto the stand in a 2-wide and 2-high configuration as shown at FIGS. 8B and 8C.

Accordingly, the inventive stand 10 can allow a user to securely position between up to four individual light therapy devices in a 2-high and/or 2-wide configuration and to selectively raise and lower the height of the same to any desired position, and to easily move the entire system from one location to another without removing, disconnecting, or adjusting the position of the light therapy devices individually.

As to a further description of the manner and use of the present invention, the same should be apparent from the above description. Accordingly, no further discussion relating to the manner of usage and operation will be provided.

As described herein, one or more elements of the light therapy device stand 10 can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A light therapy device stand, comprising:
a base having center platform, and a pair of side rails extending outward therefrom;
a riser section that extends upward from the base; and
an attachment assembly that includes a mounting plate that is connected to the riser section, and a pair of connection rails that are removably connected to the mounting plate,
wherein each of the pair of connection rails are configured to engage a back surface of at least one light therapy device and to secure the at least one light therapy device onto the mounting plate.

2. The stand of claim 1, wherein the riser section is configured to transition between an extended position and a retracted position.

3. The stand of claim 2, wherein in the extended position each of the at least one light therapy device is moved away from the base, and in the retracted position each of the at least one light therapy device is moved toward the base.

4. The stand of claim 1, wherein the riser section includes at least one fixed bar member having a first end that is connected to the center platform, an open second end, and a hollow interior space: and
at least one sliding bar member that is connected to the at least one fixed bar member.

5. The stand of claim 4, wherein the at least one sliding bar member is telescopically connected to the at least one fixed bar member.

6. The stand of claim 1, further comprising:
a plurality of openings that are positioned along each of the pair of connection rails.

7. The stand of claim 6, wherein the at least one light therapy device comprises a single light therapy device, and
wherein the plurality of openings are positioned at a location that is complementary to a location of a plurality of mounting apertures located along the back surface of the single light therapy device.

8. The stand of claim 6, wherein the at least one light therapy device comprises a first light therapy device and a second light therapy device, and
wherein the plurality of openings are positioned at a location that is complementary to a location of a plurality of mounting apertures located along the back surface of each of the first light therapy device and the second light therapy device stacked in a 2-high configuration.

9. The stand of claim 6, wherein the at least one light therapy device comprises a first light therapy device and a second light therapy device, and
wherein the plurality of openings are positioned at a location that is complementary to a location of a plurality of mounting apertures located along the back surface of each of the first light therapy device and the second light therapy device stacked in a 2-wide configuration.

10. The stand of claim 6, wherein the at least one light therapy device comprises a first light therapy device, a second light therapy device, a third light therapy device and a fourth light therapy device, and
wherein the plurality of openings are positioned at a location that is complementary to a location of a plurality of mounting apertures located along the back surface of each of the first light therapy device, the second light therapy device, the third light therapy device and the fourth light therapy device stacked in a 2-wide configuration and a 2-high configuration.

11. The stand of claim 1, wherein the base defines an unobstructed treatment area located perpendicular to the center platform and parallel to the pair of side rails.
